(19) United States
(12) Patent Application Publication
(10) Pub. No.: US 2025/0099083 A1
(43) Pub. Date: Mar. 27, 2025

(54) SCANNING CONTROL METHOD AND APPARATUS FOR ULTRASOUND SCANNING SYSTEM, AND ULTRASOUND SCANNING SYSTEM

(71) Applicant: GE Precision Healthcare LLC, Waukesha, WI (US)

(72) Inventors: Hongjian Jiang, Wuxi (CN); Siying Wang, Wuxi (CN)

(21) Appl. No.: 18/889,284

(22) Filed: Sep. 18, 2024

(30) Foreign Application Priority Data
Sep. 27, 2023 (CN) 202311271182.1

(51) Int. Cl. A61B 8/00; G06F 40/279; G16H 30/20

(52) U.S. Cl. CPC A61B 8/54; G16H 30/20; G06F 40/279

(57) ABSTRACT

A scanning control method and apparatus for an ultrasound scanning system, and an ultrasound scanning system including: acquiring an ultrasound scanning-related document, comprising an ultrasound scanning guidance for at least one tissue to be scanned; processing the ultrasound scanning-related document using a natural language processing (NLP) model, comprising processing the ultrasound scanning guidance; and generating an ultrasound scanning protocol for the at least one tissue to be scanned on the basis of the processing the ultrasound scanning guidance, and configuring and displaying the ultrasound scanning protocol in the ultrasound scanning system.

### CROSS-REFERENCE TO RELATED APPLICATION

This application claim priority to Chinese Patent Application No. 202311271182.1, which was file on Sep. 27, 2023 at the Chinese Patent Office. The entire contents of the above-listed application are incorporated by reference herein in their entirety.

### TECHNICAL FIELD

Embodiments of the present disclosure relate to the technical field of ultrasonic scanning, and in particular, to a scanning control method and apparatus for an ultrasound scanning system, and an ultrasound scanning system.

### BACKGROUND

In the field of ultrasound, in order to achieve a comprehensive and accurate diagnosis in a conventional scan, it is suggested that a sonologist follows a guideline to perform a scan proposed by the official society of medicine. There is also some need in research, where a user wishes to repeat an experiment published in a research paper using an ultrasonic scanner. For these two use scenarios, there are many common/unique operations that need to be followed, as well as a large number of detailed scanning steps, and the entire scanning process may be very complex.

It should be noted that the above introduction of the background is only for the convenience of clearly and completely describing the technical solutions of the present application, and for the convenience of understanding for those skilled in the art.

### SUMMARY

With regards to the above problems pointed out in the Background or other similar problems, embodiments of the present application provide a scanning control method and apparatus for an ultrasound scanning system, and an ultrasound scanning system.

According to an aspect of the embodiments of the present application, provided is a scanning control method for an ultrasound scanning system, comprising:
acquiring an ultrasound scanning-related document, comprising an ultrasound scanning guidance for at least one tissue to be scanned;
processing the ultrasound scanning-related document using a natural language processing (NLP) model, comprising processing the ultrasound scanning guidance; and
generating an ultrasound scanning protocol for the at least one tissue to be scanned on the basis of the processing the ultrasound scanning guidance, and configuring and displaying the ultrasound scanning protocol in the ultrasound scanning system.

According to another aspect of the embodiments of the present application, provided is a scanning control apparatus for an ultrasound scanning system, comprising:
an acquisition unit, which acquires an ultrasound scanning-related document, including an ultrasound scanning guidance for at least one tissue to be scanned;
a processing unit, which processes the ultrasound scanning-related document using a natural language processing (NLP) model, comprising processing the ultrasound scanning guidance; and
a configuration unit, which generates an ultrasound scanning protocol for the at least one tissue to be scanned on the basis of processing the ultrasound scanning guidance, and configures and displays the ultrasound scanning protocol in the ultrasound scanning system.

According to yet another aspect of the embodiments of the present application, provided is an ultrasound scanning system, comprising:
a probe, configured to collect ultrasound data;
a memory, having a natural language processing (NLP) model stored therein;
a processor, configured to perform the method according to any one of the foregoing embodiments; and
a display device, configured to receive a signal from the processor for display.

According to still another aspect of the embodiments of the present application, provided is a non-transitory computer-readable medium, having a computer program stored thereon, the computer program having at least one code segment, characterized in that, the at least one code segment is executable by a machine to cause the machine to perform steps of the method according to any one of the foregoing embodiments.

One of the beneficial effects of the embodiments of the present application is that: According to the embodiments of the present application, it is possible to facilitate understanding of scanning and analysis steps in the ultrasound scanning-related document by a user, to minimize the time taken to create a scanning template, to obtain guidance more easily with the help of an ultrasound scanner, and to know how to act in a more accurate manner, thereby reducing variables in scanning and diagnosis.

With reference to the following description and drawings, specific implementations of the embodiments of the present application are disclosed in detail, and the means by which the principles of the embodiments of the present application can be employed are illustrated. It should be understood that the embodiments of the present application are therefore not limited in scope. Within the scope of the spirit and clauses of the appended claims, the embodiments of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of an ultrasound scanning system according to an embodiment of the present application;

FIG. 2 is a schematic diagram of a scanning control method for an ultrasound scanning system according to an embodiment of the present application;

FIG. 3 is a schematic diagram of one example of acquiring an ultrasound scanning-related document by an ultrasound scanning system;

FIG. 4 is a schematic diagram of extracting the content of an ultrasound scanning guidance by an NLP model;

FIG. 5 is a schematic diagram of an ultrasound scan image 50 during an ultrasound scan;

FIG. 6 is a schematic diagram of another ultrasound scan image 60 during an ultrasound scan;

FIG. 7 is a schematic diagram of yet another ultrasound scan image 70 during an ultrasound scan;

FIG. 8 is a schematic diagram of still another ultrasound scan image 80 during an ultrasound scan; and

FIG. 9 is a schematic diagram of a scanning control apparatus for an ultrasound scanning system according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements with respect to naming, but do not represent a spatial arrangement, a temporal order, or the like of these elements, and these elements should not be limited by these terms. The term “and/or” includes one of associated listed terms or any and all combinations of a plurality of associated listed terms. The terms “comprise”, “include”, “have”, etc., indicate the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The term “include/comprise” when used herein refers to the presence of features, integrated components, steps, or assemblies, but does not preclude the presence or addition of one or more other features, integrated components, steps, or assemblies.

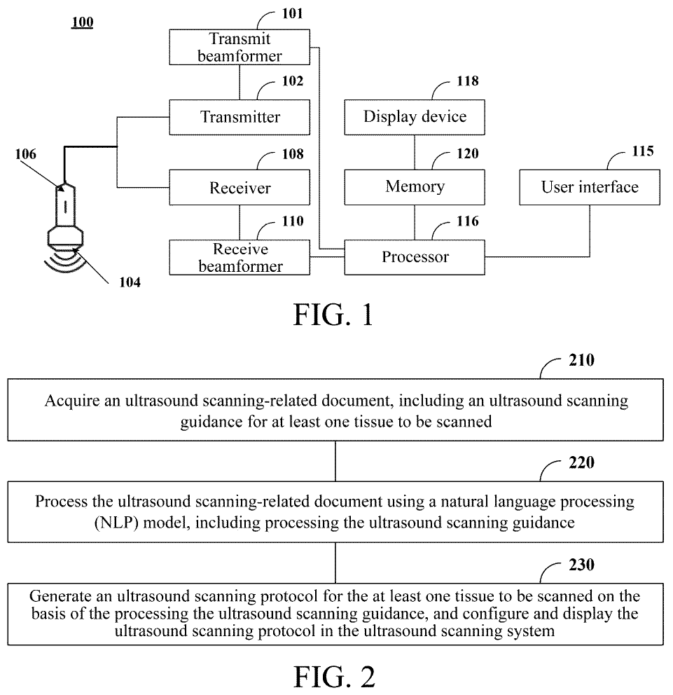

FIG. 1 is a schematic diagram of an ultrasound scanning system according to an embodiment of the present application. As shown in FIG. 1, the ultrasound scanning system 100 includes:
a transmit beamformer 101 and a transmitter 102, both driving elements 104 within a probe 106 to transmit pulsed ultrasound signals into a subject's body. The probe 106 may be any type of probe, including a linear probe, a curved array probe, a 1.25D array probe, a 1.5D array probe, a 1.75D array probe, or a 2D array probe. In addition, the probe 106 may also be a mechanical probe, for example, a mechanical 4D probe or a hybrid probe.

In the embodiments of the present application, the probe 106 may be configured to collect 4D ultrasound data, which 4D ultrasound data contains information related to how volumes change over time. Each volume may include a plurality of 2D images or slices. Still with reference to FIG. 1, the pulsed ultrasound signals are backscattered from structures within the subject's body (for example, blood cells or muscle tissues) and produce echoes that return to the elements 104. The echoes are converted by the elements 104 into electrical signals or ultrasound data, and the electrical signals are received by a receiver 108. The electrical signals representing the received echoes pass through a receive beamformer 110 that outputs ultrasound data.

According to some embodiments, the probe 106 may include an electronic circuit so as to perform all or part of transmit beamforming and/or receive beamforming. For example, all or part of the transmit beamformer 101, the transmitter 102, the receiver 108, and the receive beamformer 110 may be located in the probe 106.

In the embodiments of the present application, the terms “scan” or “scanning” may also be used to refer to collecting data through the process of transmitting and receiving ultrasound signals. The terms “data” and “ultrasound data” may be used to refer to one or more datasets collected using an ultrasound scanning system.

In the embodiments of the present application, a user interface 115 may be configured to control the operation of the ultrasound scanning system 100. The user interface 115 may be configured to control the input of the subject's data or to select various modes, operations, parameters, and so on. The user interface 115 may include one or more user input devices, for example, a keyboard, hard keys, a touch pad, a touch screen, a trackball, a rotary control, a slider, soft keys, or any other user input device.

In the embodiments of the present application, as shown in FIG. 1, the ultrasound scanning system 100 further includes a processor 116, which controls the transmit beamformer 101, the transmitter 102, the receiver 108, and the receive beamformer 110. The receive beamformer 110 may be a conventional hardware beamformer or software beamformer. If the receive beamformer 110 is a software beamformer, it may include one or more of the following components: a graphics processing unit (GPU), a microprocessor, a central processing unit (CPU), a digital signal processor (DSP), or any other type of processor capable of performing logical operations. In addition, the receive beamformer 110 may be configured to implement conventional beamforming techniques and techniques such as retrospective transmit beamforming (RTB).

In the embodiments of the present application, the processor 116 may be in electronic communication with the probe 106. For example, the processor 116 may control the probe 106 to collect ultrasound data. As another example, the processor 116 may control which elements 104 are activated and control the shape of a beam transmitted from the probe 106. In addition, the processor 116 may also be in electronic communication with the display device 118. For example, the processor 116 may process the ultrasound data into an image for display on the display device 118.

For the purpose of the present disclosure, the term “electronic communication” may be defined to include wired connection and wireless connection. According to an embodiment, the processor 116 may include a central processing unit (CPU). According to other embodiments, the processor 116 may include other electronic components capable of performing processing functions, for example, a digital signal processor (DSP), a field-programmable gate array (FPGA), a graphics processing unit (GPU), or any other type of processor. According to other embodiments, the processor 116 may include a plurality of electronic components capable of performing processing functions. For example, the processor 116 may include two or more electronic components selected from a list including the following electronic components: a central processing unit (CPU), a digital signal processor (DSP), a field-programmable gate array (FPGA), and a graphics processing unit (GPU). According to another embodiment, the processor 116 may include a complex demodulator (not shown), which demodulates RF data and generates raw data. In another embodiment, the demodulation may be performed earlier in a processing chain. The processor 116 may be adapted to perform one or a plurality of processing operations on data according to a plurality of selectable ultrasound modalities. As echo signals are received, data may be processed in real time in a scanning stage.

For the purpose of the present disclosure, the term “real time” is defined to include a process that is performed without any intentional delay. A real-time frame or volume rate may vary on the basis of a part in which data thereof is acquired or the size of a volume and specific parameters used in the collection process. The data may be temporarily stored in a buffer (not shown) in the scanning stage, and processed in a less real-time manner in live or offline operations.

The embodiments of the present application may include a plurality of processors so as to handle processing tasks. For example, a first processor may be configured to acquire an ultrasound scanning-related document, and a second processor may be configured to process the ultrasound scanning-related document using an NLP model and generate an ultrasound scanning protocol for at least one tissue to be scanned, and then configure and display the ultrasound scanning protocol in the ultrasound scanning system. It should be recognized that other embodiments may use different processor arrangements. For example, in further embodiments, the above operations may be done by the same processor. For embodiments in which the receive beamformer 110 is a software beamformer, the aforementioned processing tasks belonging to the processor 116 and the software beamformer may be performed by a single processor, for example, the receive beamformer 110 or the processor 116. Alternatively, the processing functions belonging to the processor 116 and the software beamformer may be distributed differently among any number of individual processing components.

According to an embodiment, the ultrasound scanning system 100 may continuously collect ultrasound data at a frame rate of, for example, 10 Hz to 30 Hz. An image generated from said data may be refreshed at a similar frame rate. Data may be collected and displayed at different rates in other embodiments. For example, depending on the size of the volume and potential applications, ultrasound data may be collected at a frame rate of less than 10 Hz or greater than 30 Hz in some embodiments. For example, many applications relate to collecting ultrasound data at a frame rate of 50 Hz. A memory 120 is included therein to store processing frames for collecting data. In an exemplary embodiment, the memory 120 has sufficient capacity to store ultrasound data frames that are collected over a period of time and that are at least a few seconds long. The data frames are stored by using a means which facilitates retrieval according to the order or time of the collection thereof. The memory 120 may include any known data storage medium. In addition, the memory 120 may also store an NLP model, so that the processor 116 uses the NLP model to process the ultrasound scanning-related document.

Optionally, the embodiments of the present application may be carried out by using a contrast agent. When an ultrasound contrast agent including microbubbles is used, enhanced images of anatomical structures and blood flow in the body are generated by contrast imaging. After data is collected by using a contrast agent, image analysis includes: separating a harmonic component from a linear component, enhancing the harmonic component, and generating an ultrasound image by using the enhanced harmonic component. Separation of the harmonic component from the received signal is performed by using an appropriate filter. The use of a contrast agent in ultrasonic imaging is well known to those skilled in the art, and is therefore not described in further detail.

In the embodiments of the present application, data may be processed by the processor 116 by means of modules of other or different related modes (e.g., B-mode, color Doppler, M-mode, color M-mode, spectral Doppler, elastography, TVI, strain, strain rate, etc.) to form 2D or 3D images. For example, one or more modules may generate B-mode, color Doppler, M-mode, color M-mode, spectral Doppler, elastography, TVI, strain, strain rate, a combination thereof, etc. In the embodiments of the present application, image bundles and/or frames are stored, and timing information indicating the time when the data is collected in the memory may be recorded. The module may include, for example, a scan conversion module that performs scan conversion operations so as to convert image frames from a coordinate bundle space to display space coordinates. In the embodiments of the present application, a video processor module may also be provided that reads image frames from the memory and displays in real time the image frames while performing an operation on a patient. The video processor module may store image frames in an image memory, read images from the image memory, and display the images.

In the embodiments of the present application, the ultrasound scanning system 100 may be a console-based system, a laptop computer, a hand-held or portable system, or any other configuration.

The embodiments of the present application will be described below by taking the ultrasound scanning system shown in FIG. 1 as an example, but the present application is not limited thereto, and the embodiments of the present application may also be applied to other ultrasound scanning systems.

Embodiments of the present application provide a scanning control method for an ultrasound scanning system. FIG. 2 is a schematic diagram of a scanning control method for an ultrasound scanning system according to an embodiment of the present application. As shown in FIG. 2, the method includes:
210: acquiring an ultrasound scanning-related document, including an ultrasound scanning guidance for at least one tissue to be scanned;
220: processing the ultrasound scanning-related document using a natural language processing (NLP) model, including processing the ultrasound scanning guidance; and
230: generating an ultrasound scanning protocol for the at least one tissue to be scanned on the basis of the processing the ultrasound scanning guidance, and configuring and displaying the ultrasound scanning protocol in the ultrasound scanning system.

It should be noted that FIG. 2 merely schematically illustrates the embodiments of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 2.

According to the above embodiments, by processing the ultrasound scanning-related document using the NLP model to obtain the ultrasound scanning protocol for the at least one tissue to be scanned, and then configuring and displaying the protocol in the ultrasound scanning system, it is possible to facilitate understanding of scanning and analysis steps in the ultrasound scanning-related document by a user, to minimize the time taken to create a scanning template, to obtain guidance more easily with the help of an ultrasound scanner, and to know how to act in a more accurate manner, thereby reducing variables in scanning and diagnosis. In addition, after the ultrasound scanning-related document is processed by the NLP model, the scanning template may be automatically generated on the basis of the result of the processing. Conventional scanning template configurations rely primarily on manual selection of a fixed workflow built into the ultrasound system. In contrast, the embodiments of the present application have greater flexibility and immediacy.

In operation 210, the ultrasound scanning-related document is, for example, a user manual, an ultrasound scanning-related guideline, an ultrasound scanning-related paper, or the like, and includes an ultrasound scanning guidance for the at least one tissue to be scanned (e.g., breast, abdominal cavity, and the like). These documents have relatively complex contents, which are not necessarily dedicated for scanning guidance, but merely interim research results in the field of ultrasound scanning. According to the embodiments of the present application, the relevant contents for scanning guidance may be found from these documents and refined using the NLP model.

In the embodiments of the present application, the format of the ultrasound scanning-related document is not limited, and may be in a PDF format, a WORD format, or the like as long as the document can be used for refining the text. In addition, the ultrasound scanning-related document may be from a removable storage medium, for example, a USB disk, a removable hard disk, or the like, or from a local area network, a cloud server, or the like, which is not limited in the present application.

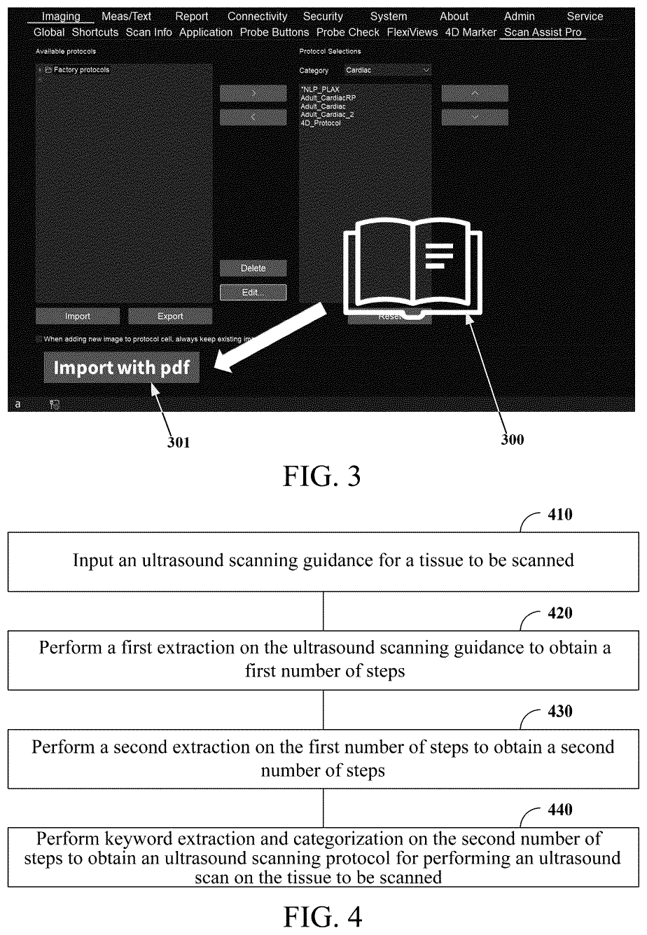

FIG. 3 is a schematic diagram of one example of acquiring an ultrasound scanning-related document by an ultrasound scanning system. As shown in FIG. 3, in this example, the ultrasound scanning-related document 300 is an ultrasound scanning-related guideline in a PDF format. The ultrasound scanning-related document 300 is acquired by means of a document input button 301, for example, called “import with pdf”, provided in the ultrasound scanning system.

In operation 220, after the ultrasound scanning-related document is acquired, the ultrasound scanning-related document may be processed using the NLP model. For example, the ultrasound scanning guidance for the at least one tissue to be scanned included in the ultrasound scanning-related document may be processed to obtain the ultrasound scanning protocol for the at least one tissue to be scanned.

In the above embodiments, the ultrasound scanning guidance may be processed by performing extraction on the content of the ultrasound scanning guidance at least once by the NLP model, and generating several steps for ultrasound scanning of the at least one tissue to be scanned.

FIG. 4 is a schematic diagram of extracting the content of an ultrasound scanning guidance by an NLP model. In the example of FIG. 4, a case in which the content of an ultrasound scanning guidance is extracted twice is shown. As shown in FIG. 4, the process includes:
410: inputting an ultrasound scanning guidance for a tissue to be scanned;
420: performing a first extraction on the ultrasound scanning guidance to obtain a first number of steps;
430: performing a second extraction on the first number of steps to obtain a second number of steps; and
440: performing keyword extraction and categorization on the second number of steps to obtain an ultrasound scanning protocol for performing an ultrasound scan on the tissue to be scanned.

For example, in operation 410, an ASE (American Society of Echocardiography) guideline is input into the NLP model. The guideline contains an ultrasound scanning guidance for an adult's heart, for a comprehensive transthoracic echocardiogram on an adult. In operation 420, a first extraction is performed on a portion of the original content of the ASE guideline using the NLP model to obtain ten steps for a transthoracic cardiac ultrasound scan. In operation 430, a second extraction is performed on the ten steps using the NLP model to obtain five steps for the transthoracic cardiac ultrasound scan. Thereafter, in operation 440, keyword extraction and categorization may be performed on the five steps using the NLP model according to preset categories, thereby obtaining an ultrasound scanning protocol for performing the transthoracic cardiac ultrasound scan.

It should be noted that FIG. 4 merely schematically illustrates the embodiments of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 4. For example, operation 430 above is optional and, in some embodiments, operation 430 may not be included such that, in operation 440, keyword extraction and categorization are performed on the first number of steps obtained in operation 420, and so on and so forth.

In the example of FIG. 4, performing extraction twice is taken as an example, but the present application is not limited thereto. Other numbers of instances of extraction, for example, only performing extraction once, or performing extraction more than twice, may also be performed as needed, for example, performed depending on different tissues to be scanned, different lengths of ultrasound scanning-related documents, or the like. In addition, in the example of FIG. 4, obtaining ten steps through the first extraction and five steps through the second extraction is taken as an example, which is likewise not limited in the present application. Other numbers of steps may be obtained for each instance of extraction depending on different ultrasound scanning-related documents and/or different tissues to be scanned.

In the above embodiments, the NLP model may perform abstractive summarization, and generate a summary by paraphrasing. This is similar to writing a summary containing words and sentences that do not appear in the source text. The abstractive summarization is typically modeled as a sequence-to-sequence task, where the input is long-format text and the output is a summary.

In the embodiments of the present application, the training and the structure of the NLP model are likewise not limited. For example, the NLP model may be a ULMFiT model, a Transformer model, a BERT model, a Transformer-XL model, a GPT-2 model, an ELMo model, a Flair model, a StanfordNLP model, and the like, for details of which reference may be made to the related technologies.

In some embodiments, the processing the ultrasound scanning guidance may also include further extracting, augmenting, or modifying the several steps in response to being operated.

For example, after the several steps are obtained, the several steps may be further extracted by the NLP model according to a user's operation, to refine the several steps. Alternatively, after the several steps are obtained, the several steps may be augmented by the NLP model according to a user's operation, to augment the several steps. Alternatively, after the several steps are obtained, a user may modify the several steps according to the user's operation, for example, adjust the order of the steps, adjust the description of the steps, and the like, so as to improve the several steps.

The aforementioned processing (performing extraction on the content of the ultrasound scanning guidance at least once) by the NLP model may not meet the requirements of the user, and the further processing (extraction, augmentation, or modification) according to this embodiment can be such that the obtained steps meet the requirements of the user, improving the flexibility in ultrasound scanning configuration.

In some embodiments, the processing the ultrasound scanning guidance may further include extracting keywords in each step, categorizing the keywords among preset categories, or categorizing each step among preset categories, to obtain categorized keywords or steps. Since the NLP model may perform named entity identification, by means of named entity identification, it may automatically scan a complete document, extract basic elements from the text, and categorize the basic elements, so as to perform relevant configuration and/or display of the ultrasound scanning system.

In the above embodiments, the preset categories may include at least one of: a target tissue, probe movement, and configuring an ultrasound scanning parameter, but the present application is not limited thereto. Other categories may also be set depending on different target tissues or different ultrasound scanning systems, so as to perform configuration and/or display related to the ultrasound scan using the categorized keywords or steps.

Taking obtaining ten steps through the first extraction of operation 420 and not performing the second extraction on the ten steps as an example, according to the above embodiments, in operation 440, the NLP model may perform keyword extraction on the ten steps according to the preset categories, and categorize the extracted keywords into respective categories. For example, the preset categories include three categories, which are a target tissue, probe movement, and configuring ultrasound scanning parameter, respectively. According to the above embodiments, from the ten steps obtained from the first extraction, the NLP model may extract keywords belonging to the three categories, and categorize them into respective categories. For example, the target tissue includes 18 keywords, the probe movement includes 3 keywords, and the ultrasound scanning parameter configuration include 5 keywords.

According to the above embodiments, on the basis of the extracted contents, i.e. the abstracted contents, the user's operations during the ultrasound scan may be categorized into different categories, which categories may encompass the target tissue in the scan and the desired operation to be performed for each step by the user. Thus, the user can know how to use the probe and buttons and expected contents in the scan.

In operation 230, the ultrasound scanning protocol, for example, includes several steps for ultrasound scanning of the at least one tissue to be scanned. That is, on the basis of the processing the ultrasound scanning guidance, several steps for ultrasound scanning of the at least one tissue to be scanned are generated. In some embodiments, a step in the ultrasound scanning protocol to which a current ultrasound scan corresponds may further be determined according to the several steps of ultrasound scanning of the at least one tissue to be scanned, and scanning assistance may be provided for the current ultrasound scanning according to the determined step.

For example, on the basis of the processing the ultrasound scanning guidance, several steps for ultrasound scanning of a tissue A to be scanned and several steps for ultrasound scanning of a tissue B to be scanned are generated. In the above embodiments, the current ultrasound scan is a scan for the tissue A to be scanned. Thus, the several steps of the ultrasound scanning of the tissue A to be scanned may be taken as a step in the ultrasound scanning protocol to which the current ultrasound scan corresponds, and scanning assistance may be provided for the current ultrasound scan according to the step.

In the above embodiments, the scanning assistance is provided for the current ultrasound scan, for example, by: in response to the corresponding step being related to configuring an ultrasound scanning parameter, automatically configuring the ultrasound scanning system; or, in response to the corresponding step being related to probe movement, providing and displaying a guide for the probe movement; or, in response to the corresponding step being related to a target tissue, automatically identifying and evaluating an image of the current ultrasound scan.

In the above embodiments, one step may include multiple categories (e.g., a target tissue, probe movement, and configuring an ultrasound scanning parameter). Therefore, multiple operations, for example, auto-configuration, guiding, and image identification, may be included in one step.

In the above embodiments, the image of the current ultrasound scan may be automatically identified and evaluated using an artificial intelligence (AI) model. In the present application, the type of the AI model is not limited, and may include, for example, various algorithms such as machine learning and depth learning as long as it can mainly perform identification and quality evaluation on the image.

According to the above embodiments, it is possible, according to the steps in the ultrasound scanning protocol to which the current ultrasound scan corresponds, to automatically configure ultrasound scanning parameters, and/or to provide a guide for movement of the probe, and/or to automatically identify and evaluate the ultrasound scan image. Thus, the workload can be reduced as much as possible. Detailed description is further provided below with reference to FIGS. 5-8, which show a series of schematic diagrams for ultrasound scanning using a scanning protocol generated by an NLP model.

FIG. 5 is a schematic diagram of an ultrasound scan image 50 during an ultrasound scan. As shown in FIG. 5, according to the method of the embodiments of the present application, a reference image 501 of anatomical features (LV, RV, Ao, and LA) to which the scanned tissue corresponds may be displayed on the ultrasound scan image 50, which is convenient for a sonologist to check whether the current ultrasound scan image contains the anatomical features.

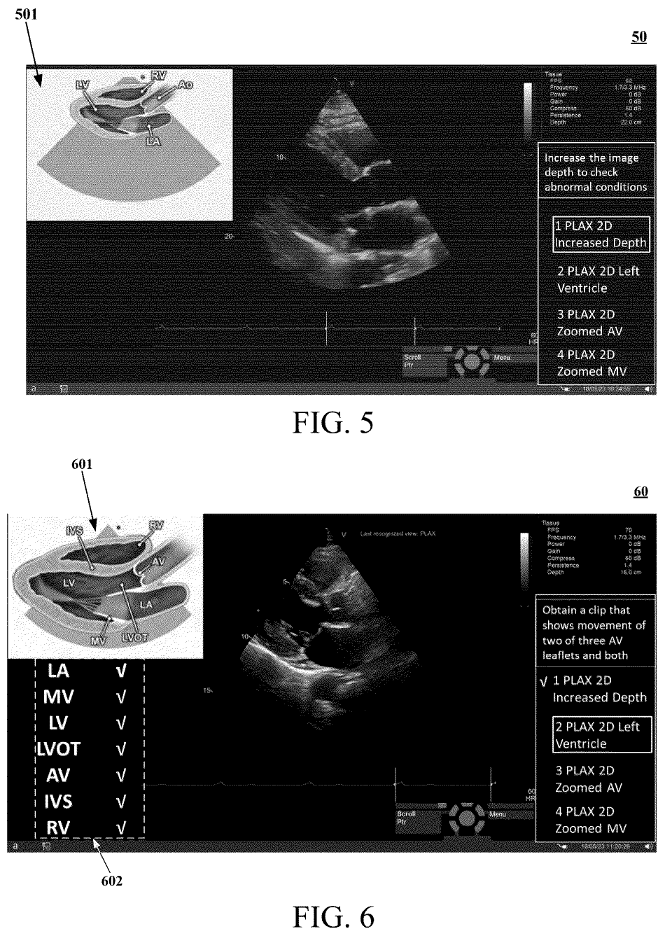

FIG. 6 is a schematic diagram of another ultrasound scan image 60 during an ultrasound scan. As shown in FIG. 6, according to the method of the embodiments of the present application, a reference image 601 of anatomical features (LA, MV, LV, LVOT, AV, IVS, and RV) to which the scanned tissue corresponds may be displayed on the ultrasound scan image 60. In addition, according to the method of the embodiments of the present application, the image of the current ultrasound scan may also be automatically identified and evaluated, for example, by automatically examining and checking whether the ultrasound scan image contains the anatomical features schematically shown by the reference image 601. The results of the examining and checking may be displayed together on the current ultrasound scan image by check marks (see a dashed line box 602), which is thus convenient for a sonologist to directly snip an ultrasound scan image consistent with the anatomical features on the reference image during the ultrasound scan.

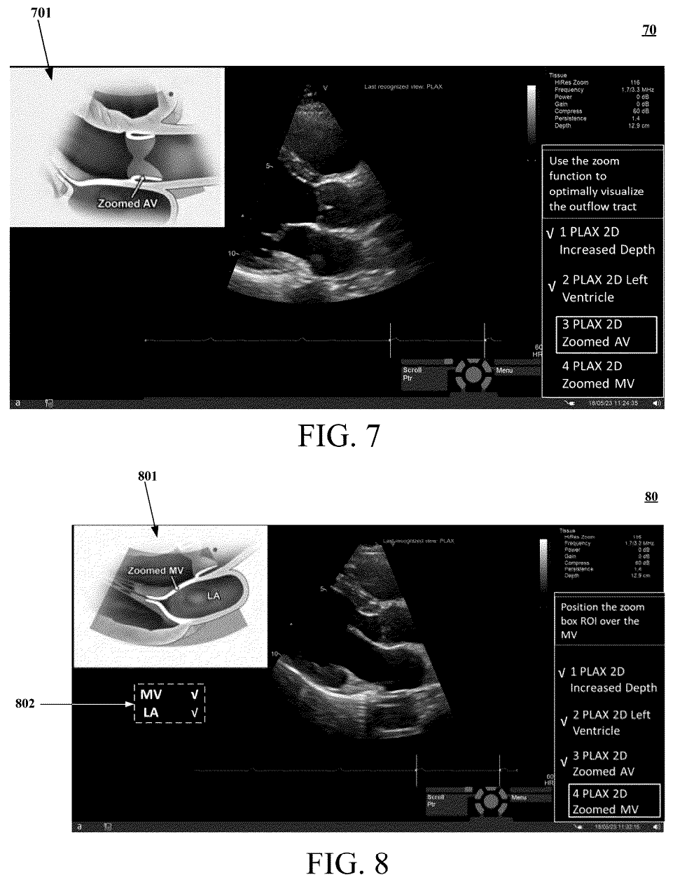

FIG. 7 is a schematic diagram of yet another ultrasound scan image 70 during an ultrasound scan. As shown in FIG. 7, according to the method of the embodiments of the present application, a reference image 701 of anatomical features (AV) to which the scanned tissue corresponds may be displayed on the ultrasound scan image, which is convenient for a sonologist to check whether the current ultrasound scan image contains the anatomical features.

FIG. 8 is a schematic diagram of still another ultrasound scan image 80 during an ultrasound scan. As shown in FIG. 8, according to the method of the embodiments of the present application, a reference image 801 of anatomical features to which the scanned tissue corresponds may be displayed on the ultrasound scan image. In addition, according to the method of the embodiments of the present application, the image of the current ultrasound scan may also be automatically identified and evaluated, for example, by automatically examining and checking whether the ultrasound scan image contains the anatomical features (MV and LA) schematically shown by the reference image 801. That is, the image of the current ultrasound scan may be automatically identified and evaluated. The results of the examining and checking may be displayed together on the current ultrasound scan image by check marks (see a dashed line box 802), which is thus convenient for a sonologist to directly snip an ultrasound scan image consistent with the anatomical features on the reference image during the ultrasound scan.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to the embodiments of the present application, by processing the ultrasound scanning-related document using the NLP model to obtain the ultrasound scanning protocol (for example, the several steps of ultrasound scanning for the at least one tissue to be scanned) for the at least one tissue to be scanned, and then configuring and displaying the protocol in the ultrasound scanning system, it is possible to facilitate understanding of scanning and analysis steps in the ultrasound scanning-related document by a user, to minimize the time taken to create a scanning template, to obtain guidance more easily with the help of an ultrasound scanner, and to know how to act in a more accurate manner, thereby reducing variables in scanning and diagnosis.

The embodiments of the present application further provide a scanning control apparatus for an ultrasound scanning system. Since the apparatus is similar to the embodiments of the first aspect in terms of principles for solving problems, reference may be made to the embodiments of the first aspect for its specific implementation, and description for the same content will not be repeated.

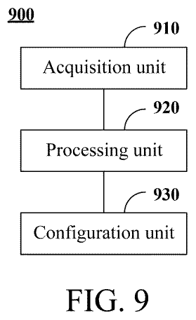

FIG. 9 is a schematic diagram of a scanning control apparatus for an ultrasound scanning system according to an embodiment of the present application. As shown in FIG. 9, a scanning control apparatus 900 for an ultrasound scanning system according to an embodiment of the present application includes:
an acquisition unit 910, which acquires an ultrasound scanning-related document, including an ultrasound scanning guidance for at least one tissue to be scanned;
a processing unit 920, which processes the ultrasound scanning-related document using a natural language processing (NLP) model, including processing the ultrasound scanning guidance; and
a configuration unit 930, which generates an ultrasound scanning protocol for the at least one tissue to be scanned on the basis of processing the ultrasound scanning guidance, and configures and displays the ultrasound scanning protocol in the ultrasound scanning system.

In some embodiments, the ultrasound scanning-related document includes at least one of a user manual, an ultrasound scanning-related guideline, and an ultrasound scanning-related paper.

In some embodiments, the ultrasound scanning-related document is from at least one of a removable storage medium, a local area network, and a cloud server.

In some embodiments, the processing the ultrasound scanning guidance by the processing unit 920 includes:
performing extraction on the content of the ultrasound scanning guidance at least once, and generating several steps for ultrasound scanning of the at least one tissue to be scanned.

In some embodiments, the processing the ultrasound scanning guidance by the processing unit 920 further includes:
in response to being operated, further extracting, augmenting, or modifying the several steps.

In some embodiments, the processing the ultrasound scanning guidance by the processing unit 920 further includes:
extracting keywords in each of the steps; and
categorizing the keywords or each of the steps among preset categories.

In the above embodiments, the preset categories may include at least one of a target tissue, probe movement, and configuring an ultrasound scanning parameter.

In some embodiments, the ultrasound scanning protocol includes several steps for ultrasound scanning of the at least one tissue to be scanned, and the configuration unit 930 may further perform the following operations:
determining a step in the ultrasound scanning protocol to which a current ultrasound scan corresponds; and
providing scanning assistance for the current ultrasound scan.

In the above embodiments, providing scanning assistance for the current ultrasound scan by the configuration unit 930 includes at least one of:
in response to the corresponding step being related to configuring an ultrasound scanning parameter, automatically configuring the ultrasound scanning system;
in response to the corresponding step being related to probe movement, providing and displaying a guide for the probe movement; and
in response to the corresponding step being related to a target tissue, automatically identifying and evaluating an image of the current ultrasound scan.

In the above embodiments, the automatically identifying and evaluating the image of the current ultrasound scan is performed using an artificial intelligence model. The manner for implementing the artificial intelligence model is not limited in the present application, for which reference may be made to the related technologies.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to the embodiments of the present application, by processing the ultrasound scanning-related document using the NLP model to obtain the ultrasound scanning protocol (for example, the several steps of ultrasound scanning for the at least one tissue to be scanned) for the at least one tissue to be scanned, and then configuring and displaying the protocol in the ultrasound scanning system, it is possible to facilitate understanding of scanning and analysis steps in the ultrasound scanning-related document by a user, to minimize the time taken to create a scanning template, to obtain guidance more easily with the help of an ultrasound scanner, and to know how to act in a more accurate manner, thereby reducing variables in scanning and diagnosis.

The embodiments of the present application further provide an ultrasound scanning system, which includes a probe, a memory, a processor, and a display device. The probe is configured to collect ultrasound data, the memory is configured for an NLP model to be stored therein, the processor is configured to perform the method according to the embodiments of the first aspect, and the display device is configured to receive a signal from the processor for display.

Since the ultrasound scanning control method according to the embodiments of the present application has been described in the embodiments of the first aspect, the contents thereof are incorporated herein and will not be described again here.

In the embodiments of the present application, the ultrasound scanning system further includes other components, for details of which reference may be made to the ultrasound scanning system shown in FIG. 1, and the same contents as those in FIG. 1 will not be described again here.

The embodiments of the present application further provide a non-transitory computer-readable medium, having a computer program stored thereon, the computer program having at least one code segment, where the at least one code segment is executable by a machine to cause the machine to perform steps of the method according to the embodiments of the first aspect.

The above apparatus and method of the present application can be implemented by hardware, or can be implemented by hardware in combination with software. The present application relates to the foregoing type of computer-readable program. When executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and variation could be easily conceived of by those skilled in the art, the embodiments of the present application are not limited to the illustrated and described precise structures and operations, but can encompass all appropriate modifications, changes, and equivalents that fall within the scope of the implementations.

1. A method for controlling an ultrasound scanning system, comprising:
acquiring an ultrasound scanning-related document, comprising an ultrasound scanning guidance for at least one tissue to be scanned;
processing the ultrasound scanning-related document using a natural language processing model, comprising processing the ultrasound scanning guidance; and
generating an ultrasound scanning protocol for the at least one tissue to be scanned on the basis of the processing the ultrasound scanning guidance, and configuring and displaying the ultrasound scanning protocol in the ultrasound scanning system.

2. The method according to claim 1, wherein
the ultrasound scanning-related document comprises at least one of a user manual, an ultrasound scanning-related guideline, and an ultrasound scanning-related paper.

3. The method according to claim 1, wherein processing the ultrasound scanning guidance comprises:
performing extraction on the content of the ultrasound scanning guidance at least once, and generating several steps for ultrasound scanning of the at least one tissue to be scanned.

4. The method according to claim 3, wherein processing the ultrasound scanning guidance further comprises:
in response to being operated, further extracting, augmenting, or modifying the several steps.

5. The method according to claim 3, wherein processing the ultrasound scanning guidance further comprises:
extracting keywords in each of the steps; and
categorizing the keywords or each of the steps among preset categories.

6. The method according to claim 5, wherein
the preset categories comprise at least one of a target tissue, probe movement, and configuring an ultrasound scanning parameter.

7. The method according to claim 1, wherein the ultrasound scanning protocol comprises several steps for ultrasound scanning of the at least one tissue to be scanned, and the method further comprises:
determining a step in the ultrasound scanning protocol to which a current ultrasound scan corresponds; and
providing scanning assistance for the current ultrasound scan.

8. The method according to claim 7, wherein providing scanning assistance for the current ultrasound scan comprises at least one of:
in response to the corresponding step being related to configuring ultrasound scanning parameter, automatically configuring the ultrasound scanning system;
in response to the corresponding step being related to probe movement, providing and displaying a guide for the probe movement; and
in response to the corresponding step being related to a target tissue, automatically identifying and evaluating an image of the current ultrasound scan.

9. The method according to claim 8, wherein the automatically identifying and evaluating an image of the current ultrasound scan is performed using an artificial intelligence model.

10. An ultrasound scanning system, characterized by comprising:
a probe, configured to collect ultrasound data;
a memory, having a natural language processing model stored therein;
a processor, configured to:
acquire an ultrasound scanning-related document, comprising an ultrasound scanning guidance for at least one tissue to be scanned;
process the ultrasound scanning-related document using a natural language processing model, comprising processing the ultrasound scanning guidance; and
generate an ultrasound scanning protocol for the at least one tissue to be scanned on the basis of the processing the ultrasound scanning guidance, and configuring and displaying the ultrasound scanning protocol in the ultrasound scanning system; and
a display device, configured to receive a signal from the processor for display.

11. The system according to claim 10, wherein
the ultrasound scanning-related document is from at least one of a removable storage medium, a local area network, and a cloud server.

12. A non-transitory computer-readable medium, having a computer program stored therein, the computer program having at least one code segment, characterized in that, the at least one code segment is executable by a machine to cause the machine to perform steps of:
acquiring an ultrasound scanning-related document, comprising an ultrasound scanning guidance for at least one tissue to be scanned;
processing the ultrasound scanning-related document using a natural language processing model, comprising processing the ultrasound scanning guidance; and
generating an ultrasound scanning protocol for the at least one tissue to be scanned on the basis of the processing the ultrasound scanning guidance, and configuring and displaying the ultrasound scanning protocol in the ultrasound scanning system.
